(19) United States
(12) Patent Application Publication
(10) Pub. No.: US 2019/0142277 A1
(43) Pub. Date: May 16, 2019

(54) PHOTOACOUSTIC APPARATUS AND OBJECT INFORMATION ACQUIRING METHOD

(71) Applicant: CANON KABUSHIKI KAISHA, Tokyo (JP)

(72) Inventors: Toshinobu Tokita, Yokohama-shi (JP); Naoto Abe, Machida-shi (JP)

(21) Appl. No.: 16/181,826

(22) Filed: Nov. 6, 2018

(30) Foreign Application Priority Data
Nov. 10, 2017 (JP) 2017-217509

(51) Int. Cl. A61B 5/00; G01N 29/24; G01N 29/04

(52) U.S. Cl. CPC A61B 5/0095; G01N 29/2418; A61B 5/14542; A61B 5/444; G01N 2291/02475; G01N 29/04

(57) ABSTRACT

A photoacoustic apparatus is configured to receive an acoustic wave generated from an object to which light is irradiated, and to generate object information which is information on the object, the apparatus includes: an irradiation control unit that controls an irradiated region of the light on the object; an acoustic wave receiving unit that receives the acoustic wave generated from the object, and converts the acoustic wave into a received signal; a generating unit that generates the object information based on the received signal; and a setting unit that sets, for the object, a high absorption region in which a value related to absorption of an optical energy is at least a predetermined value, based on the received signal, wherein the irradiation control unit controls the irradiated region of the light on the object based on a position of the high absorption region.

### BACKGROUND OF THE INVENTION

### Field of the Invention

The present invention relates to a photoacoustic apparatus that acquires object information.

### Description of the Related Art

As a technique to image functional information inside an object, such as structural information and physiological information, a photoacoustic imaging (hereafter PAI) is known.

When light, such as laser light, is irradiated to a living body (object), an acoustic wave (typically an ultrasonic wave) is generated when the light is absorbed by a biological tissue inside the object. This phenomenon is called a “photoacoustic effect”, and the acoustic wave generated by the photoacoustic effect is called a “photoacoustic wave”. The tissues constituting the object have different absorption rates of optical energy, hence the generated photoacoustic waves also have different sound pressures. With PAI, a generated photoacoustic wave is received by a probe, and the received signal is mathematically analyzed so as to acquire characteristic information inside the object.

### SUMMARY OF THE INVENTION

As an application example of the photoacoustic imaging, imaging micro-vasculature caused by cancer or inflammation is expected.

On the other hand, a region having a high optical energy absorption coefficient, such as a mole or a nevus, may exist on the surface of an object. If light is irradiated to such a region having a high absorption coefficient, a photoacoustic wave having high sound pressure is generated. Then in some cases a photoacoustic wave, of which level is much higher than that of the photoacoustic wave acquired from a micro-vasculature inside a living body, may be generated.

If a light absorber exists near the surface of a living body (object) like this, a strong acoustic wave generated near the surface is reflected or scattered inside the object, and in some cases, observation of a light absorber existing in a deep region of the object may be interfered with. For example, noise and a virtual image (artifact) may be generated in a deep region of the object due to reflected acoustic waves, and as a result, sufficient contrast may not be acquired for the observation target light absorber.

With the foregoing problem of prior art in view, it is an object of the present invention to reduce noise that is superimposed on object information in a photoacoustic image.

In order to solve the aforementioned problem, the photoacoustic apparatus according to the present invention is configured to receive an acoustic wave generated from an object to which light is irradiated, and to generate object information which is information on the object, the apparatus includes: an irradiation control unit that controls an irradiated region of the light on the object; an acoustic wave receiving unit that receives the acoustic wave generated from the object, and converts the acoustic wave into a received signal; a generating unit that generates the object information based on the received signal; and a setting unit that sets, for the object, a high absorption region in which a value related to absorption of an optical energy is at least a predetermined value, based on the received signal, wherein the irradiation control unit controls the irradiated region of the light on the object based on a position of the high absorption region.

In addition, the object information acquiring method according to the present invention is performed by a photoacoustic apparatus configured to receive an acoustic wave generated from an object to which light is irradiated, and to generate object information which is information on the object, the method includes: an irradiation control step of controlling an irradiated region of the light on the object; an acoustic wave receiving step of receiving the acoustic wave generated from the object, and converting the acoustic wave into a received signal; a generating step of generating the object information based on the received signal; and a setting step of setting, for the object, a high absorption region in which a value related to absorption of an optical energy is at least a predetermined value, based on the received signal, wherein, in the irradiation control step, the irradiated region of the light on the object is controlled based on a position of the high absorption region.

According to the present invention, noise that is superimposed on object information can be reduced in a photoacoustic image.

Further features of the present invention will become apparent from the following description of exemplary embodiments with reference to the attached drawings.

### BRIEF DESCRIPTION OF THE DRAWINGS

FIG. 1 is a diagram depicting a general configuration of a photoacoustic apparatus which is common to each embodiment;

FIG. 2 is a diagram depicting a configuration of a photoacoustic microscope according to Embodiment 1;

FIG. 3A, 3B and 3C are diagrams depicting an operation of the photoacoustic microscope according to Embodiment 1;

FIG. 4 is a flow chart depicting the content of the processing according to Embodiment 1;

FIG. 5 is a diagram depicting a configuration of a photoacoustic microscope according to Embodiment 2;

FIG. 6A, 6B and 6C are diagrams depicting an operation of the photoacoustic microscope according to Embodiment 2;

FIG. 7 is a diagram depicting a configuration of a photoacoustic microscope according to Embodiment 3;

FIG. 8 is a diagram depicting a modification of Embodiment 3;

FIG. 9 is a diagram depicting a modification of Embodiment 3;

FIG. 10A, 10B and 10C are diagrams depicting an operation of a photoacoustic microscope according to Embodiment 3;

FIG. 11 is a flow chart depicting a content of the processing according to Embodiment 4;

FIG. 12 is a flow chart depicting a content of the processing according to Embodiment 5;

FIG. 13 is a diagram depicting a difference of light quantity distribution inside the object; and

FIG. 14A, 14B and 14C are diagrams depicting apodization.

### DESCRIPTION OF THE EMBODIMENTS

Embodiments of the present invention will be described with reference to the drawings. Dimensions, materials, shapes and relative positions of the components described below should be appropriately changed depending on the configurations and various conditions of the apparatus to which the invention is applied. Therefore the following description is not intended to limit the scope of the invention.

The present invention relates to a technique to detect an acoustic wave propagating from an object, and generate and acquire the characteristic information inside the object. This means that the present invention is regarded as a photoacoustic apparatus or a control method thereof The present invention is also regarded as a program which causes the apparatus, equipped with such hardware resources as a CPU and memory, to execute these methods, or a computer readable non-transitory storage medium storing this program.

Further, the present invention can also be regarded as an information processing apparatus that processes signals acquired by the photoacoustic apparatus and the object information acquiring apparatus, and the information processing method thereof.

The photoacoustic apparatus according to the embodiments is an apparatus utilizing a photoacoustic effect, that is, an acoustic wave, generated inside an object by irradiating light (electromagnetic wave) to the object, is received, and the characteristic information of the object is acquired as the image data. In this case, the characteristic information refers to information on the characteristic values corresponding to a plurality of positions inside the object respectively, and these characteristic values are generated using the received signals which are acquired by receiving a photoacoustic wave.

The characteristic information acquired by the photoacoustic measurement refers to the values reflecting the absorption rate of the optical energy. For example, the characteristic information includes a generation source of an acoustic wave which was generated by the light irradiation, an initial sound pressure inside the object, an optical energy absorption density and an absorption coefficient derived from the initial sound pressure, and a concentration of a substance constituting a tissue.

Based on the photoacoustic waves that are generated by lights having a plurality of different wavelengths, spectral information, such as concentration of a substance constituting the object, can be acquired. The spectral information may be an oxygen saturation, a value generated by weighting the oxygen saturation by intensity (e.g. absorption coefficient), a total hemoglobin concentration, an oxyhemoglobin concentration or a deoxyhemoglobin concentration. The spectral information may also be a glucose concentration, a collagen concentration, a melanin concentration, or a volume percentage of fat or water.

In the following embodiments described below, it is assumed that a photoacoustic imaging apparatus is used to acquire data on distribution and profiles of blood vessels inside the object and data on the oxygen saturation distribution in the blood vessels, by irradiating light having a wavelength which is determined based on the assumption that the absorber is hemoglobin, to the object.

Based on the characteristic information at each position inside the object, a two-dimensional or three-dimensional characteristic information distribution is acquired. The distribution data can be generated as image data. The characteristic information may be determined, not as numeric data, but as distribution information at each position inside the object. In other words, such distribution information as the initial sound pressure distribution, the energy absorption density distribution, the absorption coefficient distribution and the oxygen saturation distribution may be determined.

The “acoustic wave” in the present description is typically an ultrasonic wave, including an elastic wave called a “sound wave” or a “photoacoustic wave”. An electric signal, converted from an acoustic wave by a probe or the like, is called an “acoustic signal”. Such phrases as “ultrasonic wave” or “acoustic wave” in this description, however, are not intended to limit the wavelengths of these elastic waves. An acoustic wave generated due to the photoacoustic effect is called a “photoacoustic wave” or a “light-induced ultrasonic wave”. An electric signal, which originates from a photoacoustic wave, is called a “photoacoustic signal”. In this description, the photoacoustic signal includes both an analog signal and a digital signal. The distribution data is also called “photoacoustic image data” or “reconstructed image data”.

The photoacoustic apparatus according to the embodiments is an apparatus that irradiates a pulsed light to an object and receives a photoacoustic wave generated inside the object, so as to generate information related to the optical characteristic inside the object.

Overview of Apparatus Configuration

An apparatus configuration common to a plurality of embodiments in this description will be described first.

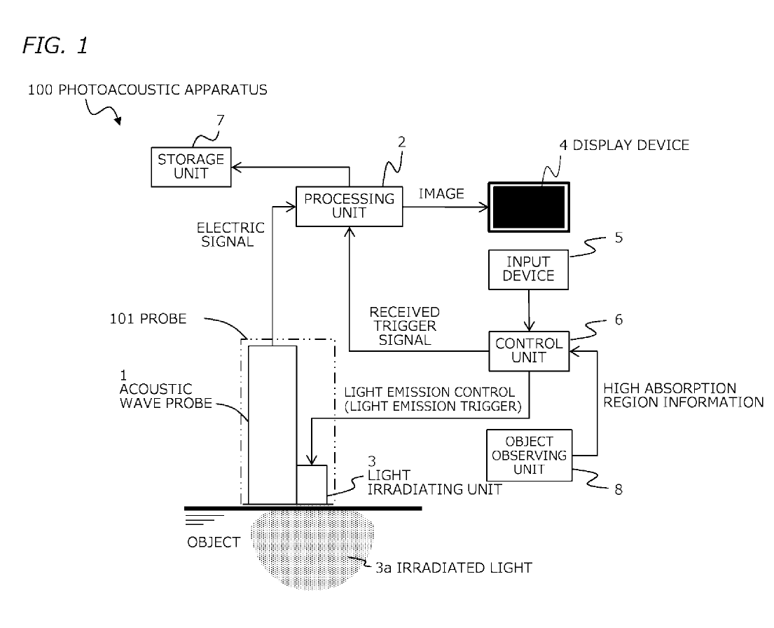

FIG. 1 is a schematic diagram depicting a configuration of a photoacoustic apparatus 100 according to an embodiment of the present invention. The photoacoustic apparatus 100 according to the embodiment is constituted of a probe 101, a processing unit 2, a display device 4, an input device 5, a control unit 6, a storage unit 7 and an object observing unit 8. The probe 101 includes an acoustic wave probe 1 and a light irradiating unit 3.

The probe 101 is a unit configured to irradiate light to an object and receive an acoustic wave generated from the object. The probe 101 includes a light irradiating unit 3 that irradiates light to the object, and an acoustic wave probe 1 that receives an acoustic wave generated in the object. The probe 101 may contact the object via an acoustic matching material (not illustrated), such as gel and water.

The acoustic wave probe 1 is a unit that receives an acoustic wave propagated from inside the object, and converts the acoustic wave into an electric signal. The acoustic wave detecting element is also called a probe, an acoustic wave probe, an acoustic wave detector or an acoustic wave receiver.

The acoustic wave generated from a living body is an ultrasonic wave in the 100 KHz to 100 MHz range, hence an element that can receive this frequency band is used for the acoustic wave detecting element. In concrete terms, an acoustic wave probe utilizing a piezoelectric phenomenon, an acoustic wave probe utilizing the resonance of light, an acoustic wave probe utilizing the change in capacitance or the like can be used.

It is preferable that the acoustic element has high sensitivity and can receive a wide frequency band. In concrete terms, a piezoelectric element using lead zirconate titanate (PZT), or an acoustic element using a high polymer piezoelectric material, such as polyvinylidene fluoride (PVDF), a capacitive micro machined ultrasonic transducer (CMUT), a Fabry-Perot interferometer or the like may be used. The acoustic element, however, is not limited to the above, but may be any element as long as the functions of the probe can be executed.

The light irradiating unit 3 is constituted by a light source configured to generate light (typically a pulsed light) which is irradiated to an object, and an optical system configured to guide this light to the probe unit.

The light source is a unit configured to generate light which is irradiated to an object. The light source is preferably a laser light source in order to generate high power, but a light-emitting diode or flash lamp may be used instead of laser. In the case of using a laser as the light source, various lasers, such as a solid-state laser (e.g. Nd:YAG, Ti:sa, OPO and alexandrite), a gas laser, a dye laser and a semiconductor laser can be used.

The wavelength of the pulsed light is preferably a specific wavelength that is absorbed by a specific component, out of the components constituting the object, and is also a wavelength by which light can propagate into the object. In concrete terms, a wavelength from 700 nm, to 1100 nm is preferable. The light in this region can reach a relatively deep region of the living body, hence information in the deep region of the object can be acquired. If the imaging region is limited to a shallow region of the object, other wavelengths, such as 532 nm, may be used.

To effectively generate the photoacoustic wave, the light must be irradiated for a sufficiently short time, in accordance with the thermal characteristics of the object. When the object is a living body, the pulse width of the pulsed light that is generated from the light source is preferably from several nano to several hundred nano seconds.

The profile of the pulsed light is preferably rectangular but may be Gaussian.

The timing, waveform, intensity and the like of the light irradiation are controlled by the later mentioned control unit 6.

The optical system is a member configured to transmit a pulsed light emitted from the light source. The light emitted from the light source is guided to the object, while being processed to a predetermined light distribution profile by such optical components as a lens and mirror, and is irradiated. The light may be propagated by an optical wave guide, such as optical fiber.

The optical system may include such optical components as a lens, a mirror, a prism, an optical fiber, a diffusion plate, a shutter and a filter. Any optical component may be used for the optical system as long as the light emitted from the light source can be irradiated in a desired profile to the object.

The processing unit 2 includes a unit that amplifies an electric signal acquired by the acoustic wave probe 1 and converts the electric signal into a digital signal, and a unit that acquires object information such as a light absorption coefficient and oxygen saturation inside the object (generating unit) based on the converted digital signal (photoacoustic signal).

The processing unit 2 may be configured using an amplifier that amplifies a received signal, an A/D convertor that converts an analog received signal into a digital signal, a memory (e.g. FIFO) that stores the received signal, and an arithmetic circuit (e.g. FPGA chip). Further, the processing unit 2 may be configured by a plurality of processors and arithmetic circuits.

The processing unit 2 generates an initial sound pressure distribution in a three-dimensional object based on the collected electric signals. The processing unit 2 also generates a three-dimensional light intensity distribution inside the object, based on the information on the quantity of light irradiated to the object. The three-dimensional light intensity distribution can be acquired by solving the light diffusion equation using information on the two-dimensional light intensity distribution. Further, the absorption coefficient distribution inside the object may be acquired using the initial sound pressure distribution inside the object generated from the photoacoustic signals and the three-dimensional light intensity distribution. Furthermore, the oxygen saturation distribution inside the object may be acquired by computing the absorption coefficient distributions at a plurality of wavelengths.

The processing unit 2 may have a function to perform a desired processing, such as information processing required for calculating the light quantity distribution, or acquiring an optical coefficient of the background, and a function to perform signal correction.

The control unit 6 performs control of each composing element of the photoacoustic apparatus 100 (irradiation control unit). For example, the control unit 6 instructs control of light irradiation to the object, reception control of the acoustic wave and photoacoustic signal, and control of the entire apparatus. The processing unit 2 and the light irradiating unit 3 perform the light irradiation and photoacoustic signal acquisition at a timing synchronizing with the trigger signal issued by the control unit 6.

The control unit 6 acquires instructions on the measurement parameters, the start/end of the measurement, the selection of the image processing method, the storing of patient information and images, and data analysis, via the input device 5, and controls the apparatus based on these instructions.

The input device 5 is, for example, a pointing device (e.g. mouse, track ball, touch panel) and keyboard, but is not limited to these.

The display device 4 displays information acquired by the processing unit 2 and the processed information thereof, and is typically a display unit.

The storage unit 7 stores object information acquired by the apparatus (photoacoustic image) and related data. The storage unit 7 may be a local storage or an external storage unit connected via a network. Further, the storage unit 7 may be a storage device including an interface with an external storage unit. For example, a computer in a medical facility may be connected via a network using an interface (not illustrated), or an external recording device (not illustrated), such as a memory and hard disk, may be connected so as to transmit the stored data.

The object observing unit 8 optically observes the surface of the object, and is typically a camera. Based on the acquired image, the object observing unit 8 detects a region in which the absorption coefficient of the optical energy is high (hereafter called “high absorption region”), such as a mole and nevus existing on the surface of the object. A specific method thereof will be described later.

A method of the photoacoustic apparatus 100 to measure a living body (object) will be described next.

First a pulsed light emitted from the light irradiating unit 3 is irradiated to the object via the optical system. When a part of the energy of the light propagating inside the object is absorbed by a light absorber (e.g. blood), an acoustic wave is generated from this light absorber by thermal expansion. If a cancer exists in a living body, light is uniquely absorbed by the newly generated blood vessels of the cancer, in the same manner as the case of blood in a normal region, and an acoustic wave is generated. The photoacoustic wave generated inside the living body is received by the acoustic wave probe 1.

The signal received by the acoustic wave probe 1 is converted and analyzed by the processing unit 2. The analysis result becomes a volume data which represents the characteristic information (e.g. initial sound pressure distribution, absorption coefficient distribution) inside the living body, and is converted into a two-dimensional image, and is then output via the display device 4.

Prior to measurement, the photoacoustic apparatus 100 detects a high absorption region in an image of the object captured by the object observing unit 8, and irradiates light to the object while avoiding the high absorption region, so that light exceeding a predetermined intensity is not irradiated to this high absorption region. The specific method of the object observing unit 8 detecting the high absorption region, and the specific method of irradiating light while avoiding the high absorption region will be described later for each embodiment.

### Embodiment 1

Embodiment 1 is an embodiment in a case where the above mentioned photoacoustic apparatus 100 is applied to a photoacoustic microscope. Description on the elements described with reference to FIG. 1 is omitted.

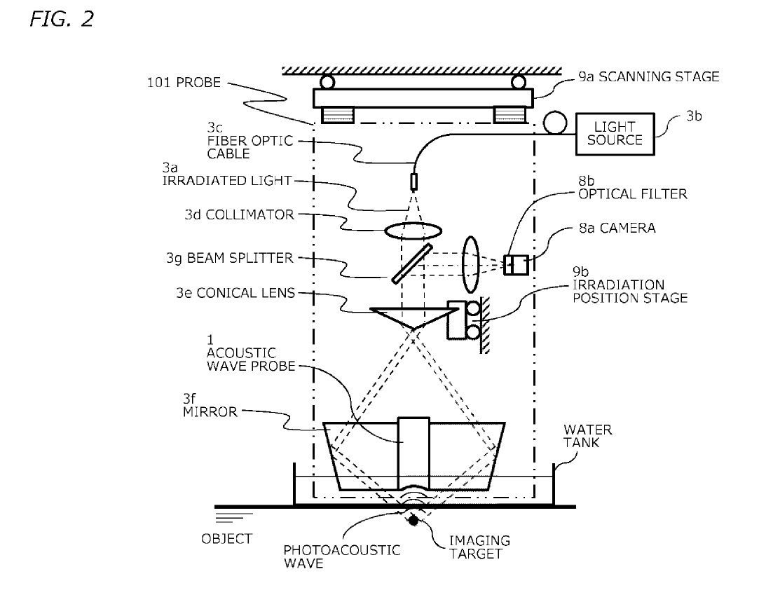

FIG. 2 is a block diagram depicting the photoacoustic microscope according to Embodiment 1.

In Embodiment 1, the light irradiating unit 3 illustrated in FIG. 1 includes a light source 3b, an optical fiber 3c, a collimator 3d, a conical lens 3e, a mirror 3f and a beam splitter 3g.

The irradiated light 3a emitted from the light source 3b is transferred to the probe 101 by the optical fiber 3c, and becomes parallel light by the collimator 3d. Then this light is spread into a ring shape by the conical lens 3e, and is irradiated to the living body (object) via the mirror 3f. The mirror 3f is a member that totally reflects the light on the side face thereof using the difference between the reflectances of a base material, which is a transparent member (e.g. glass, acryl), and air, or that reflects light by forming a metal film or dielectric film on the side face thereof.

In Embodiment 1, an acoustic lens is mounted at the tip of the acoustic wave probe 1, so that the photoacoustic wave generated at the focal position can be detected at high sensitivity and high resolution.

Further, in Embodiment 1, a scanning stage 9a is connected to the probe 101, so that the interval between the probe 101 and the object is adjusted, and two-dimensional scanning is performed on the object in the in-plane direction. By performing the two-dimensional scanning on the object, the three-dimensional photoacoustic information inside the object can be acquired.

In Embodiment 1, an image on the surface of the object is acquired using a camera 8a which is the object observing unit 8. The acquired image is binarized using a predetermined threshold, and a region in which the density is at least a predetermined value is extracted as a region in which the absorption coefficient is high (high absorption region), such as a mole and a nevus. The extracted information on the high absorption region is transferred to the control unit 6. The coordinates of the high absorption region may be expressed using a coordinate system based on the scanning stage 9a (scanning coordinate system).

In Embodiment 1, the probe 101 is constructed to be movable by the scanning stage 9a, thereby measurement can be performed while moving the light irradiation position on the object. Further, the conical lens 3e can be moved in the direction perpendicular to the object by the irradiation position stage 9b, so that the inner and outer diameters of the ring-shaped irradiated light 3a can be adjusted.

In this description, a light irradiation position is based on a concept which includes both the target point to which light is irradiated, and the location where light is irradiated (irradiated region).

In Embodiment 1, if the light irradiation position includes a part of the high absorption region, the control unit 6 detects this during measurement, and moves the irradiation position stage 9b, so that the inner and outer diameters of the ring-shaped irradiated light 3a can be adjusted to disperse the light. Thereby the intensity of the irradiated light to the region having a high absorption coefficient can be decreased. The inner and outer diameters of the ring-shaped irradiated light 3a can be adjusted so that irradiation of light to the high absorption region is avoided.

In the example in FIG. 2, the camera 8a images the surface of the object via the beam splitter 3g, but the position of the camera 8a is not limited to this. For example, the camera may be disposed in a position adjacent to the acoustic wave probe 1. It is even better if the optical filter 8b is disposed so that the irradiated light does not directly enter the camera 8a. By disposing a filter to cut the wavelength of the irradiated light as the optical filter 8b, the influence of the irradiated light on the camera 8a can be suppressed.

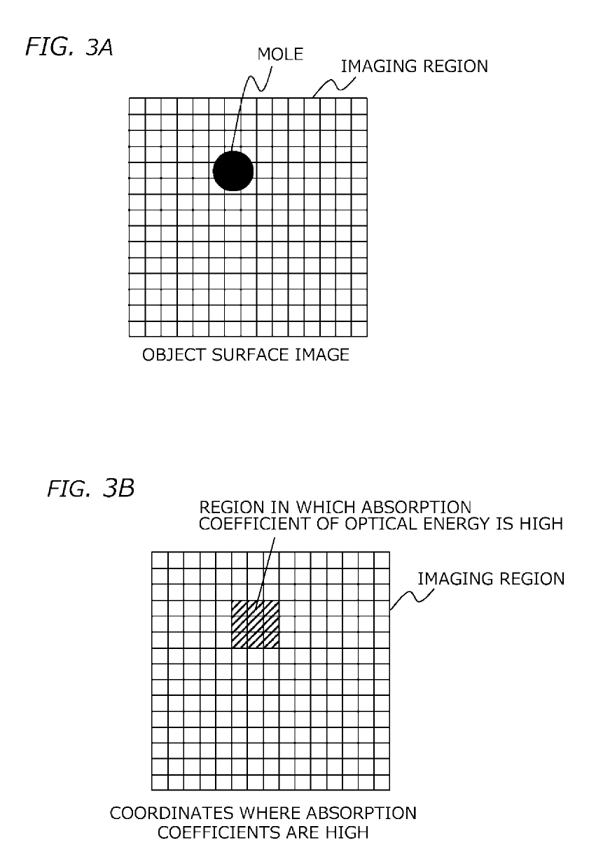

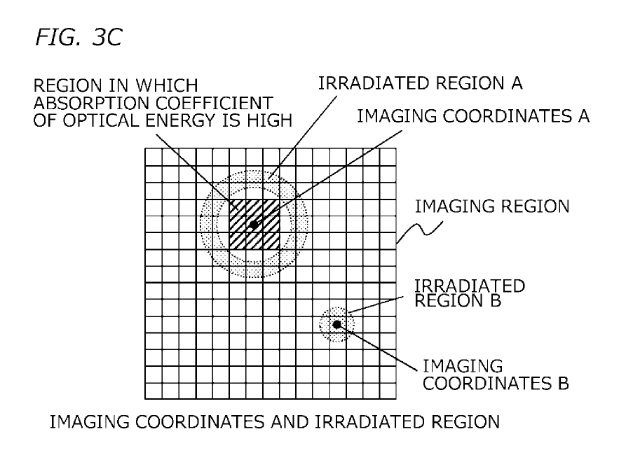

Details of the control performed by the control unit 6 according to Embodiment 1 will be described with reference to FIG. 3A to FIG. 3C.

FIG. 3A to FIG. 3C illustrate imaging regions, where the grid indicates the coordinates used for imaging (hereafter called “imaging coordinates”). If there is a mole in the imaging region on the surface, an image illustrated in FIG. 3A is captured by the camera 8a.

In the case of this example, the control unit 6 records that the high absorption region exists in the region hatched in FIG. 3B.

Here a case where the probe 101 is moved by the scanning stage 9a to perform measurement in the imaging coordinates indicated in FIG. 3C will be described. In this case, the control unit 6 controls the position of the irradiation position stage 9b to expand the irradiated region, so that the intensity of light irradiated to the high absorption region does not exceed a predetermined value. In the case of this example, the light is irradiated in the state of being dispersed in a ring-shaped region indicated by the irradiated region A.

In the case of performing measurement in the imaging coordinates B, on the other hand, the control unit 6 controls the position of the irradiation position stage 9b to narrow down the irradiated region. As a result, the light is irradiated to the irradiated region B.

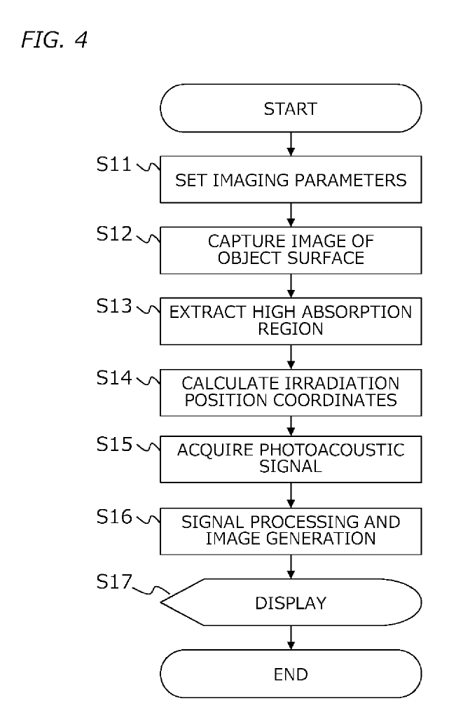

A processing flow performed by the photoacoustic microscope according to Embodiment 1 will be described next with reference to FIG. 4.

First in step S11, the imaging parameters are set. In this step, parameters to capture the photoacoustic image are acquired and set. In concrete terms, an imaging pitch and an imaging range to acquire the photoacoustic signals, a sampling frequency to store the photoacoustic signals and the storing time thereof at each imaging location, a scanning speed and acceleration of the scanning stage 9a, a light-emitting frequency, the light quantity and wavelength of the light source 3b and the like are set, and recorded in the processing unit 2 and the control unit 6. These parameters may be selected from the preset parameters, or may be input by the user of the apparatus.

Then in step S12, an image of the object surface is captured using the camera 8a. It is preferable that the imaging range includes the imaging range of the photoacoustic image. In this case, it is preferable to consider the difference between the position of the camera 8a and the position of the acoustic wave probe 1. The image captured by the camera 8a is sent to the control unit 6.

In Embodiment 1, the imaging position of the camera 8a moves in tandem with the scanning stage 9a. Therefore in step S12, the position of the scanning stage 9a may be controlled so that an image of the entire imaging range that is set can be acquired.

In step S13, a region having a high absorption coefficient is extracted from the acquired image. In this step, the control unit 6 analyzes the image captured in step S12, and extracts a tissue, of which absorbed light quantity, with respect to the light absorber (e.g. hemoglobin in blood) to be visualized, cannot be ignored, such as a mole or nevus existing on the object surface, and detects the position of the tissue in the image. In concrete terms, the control unit 6 binarizes the image captured by the camera 8a using a predetermined threshold, and extracts a region having at least a predetermined value of density, as a region having a high absorption coefficient (high absorption region), such as a mole and nevus.

Then in step S14, the target coordinates where light is irradiated (hereafter called “irradiation position coordinates”) are calculated. In this step, the control unit 6 calculates the coordinates of the irradiation position so as to decrease the intensity of the irradiated light to the high absorption region extracted in step S13. In the case of performing light irradiation for a plurality of times while moving the probe 101, a plurality of irradiation position coordinates are calculated.

Then in step S15, photoacoustic signals are acquired. In this step, the control unit 6 controls the irradiation of light to the object in accordance with the imaging parameters which were set in step S11 and the irradiation position coordinates recorded in step S14. Further, the processing unit 2 acquires the photoacoustic signals while moving the scanning stage 9a in accordance with the imaging pitch and imaging range which were set as the parameters. The photoacoustic signals are acquired at timings synchronizing with the emission of the irradiated light.

In step S16, the acquired photoacoustic signals are processed to generate the photoacoustic image.

In concrete terms, the processing unit 2 converts the acquired analog signals into digital signals using the preamplifier and A/D convertor, and stores the converted digital signals in memory. The stored data may be amplified or filtered. It is preferable that the stored data is corrected considering the impulse response characteristic of the acoustic wave probe 1.

Then the processing unit 2 generates an image based on the coordinates corresponding to the processed photoacoustic signals. To generate the image, a phasing addition (the Delay And Sum Method), which is normally used in an ultrasonic diagnostic apparatus, may be used, or an image reconstructing method, such as universal back projection, may be used. Further, such a known image processing as artifact removal may be performed.

In step S17, the generated photoacoustic image is output to the display device 4. Here the generated image may be recorded in the storage unit 7. Further, the photoacoustic image, various imaging conditions and the like may be transmitted, via the external interface, to other computers in the medical facility connected by a network, or to an external storage device (not illustrated) such as a memory and a hard disk.

As described above, according to Embodiment 1, the intensity of the irradiated light to a region having a high absorption coefficient (e.g. mole, nevus) existing on the surface in the photoacoustic measurement range can be decreased. In other words, the level of the photoacoustic wave emitted from this region can be decreased.

Further, it can be prevented that this photoacoustic wave is reflected and scattered inside the object, mixing with the photoacoustic wave generated in the original observation target (e.g. micro-vasculature), therefore the S/N ratio of the photoacoustic image can be improved. As a result, the contrast of the observation target can be improved. Furthermore, the signal generated at a position deeper than the region having a high absorption coefficient (e.g. mole, nevus) can be accurately acquired, hence an accurate photoacoustic image can be acquired from a desired imaging region.

### Embodiment 2

In Embodiment 1, the intensity of the irradiated light to the high absorption region is decreased by adjusting the inner diameter and the outer diameter of the light which is irradiated in a ring shape. In Embodiment 2, on the other hand, the irradiated light is transferred via an optical fiber, and the position of the emitting end from which the irradiate light is emitted is temporarily shifted, so that irradiation of light to the high absorption region can be avoided.

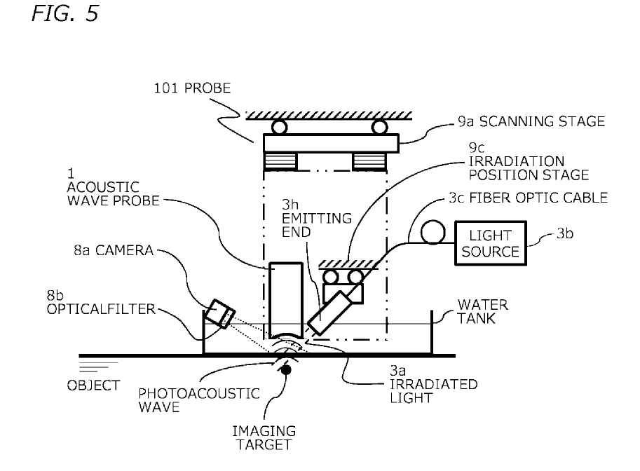

FIG. 5 is a block diagram depicting a photoacoustic microscope according to Embodiment 2.

In Embodiment 2, the light irradiating unit 3 includes a light source 3b, an optical fiber 3c and an emitting end 3h. Inside the emitting end 3h, an optical element, such as a diffusion plate, may be disposed. Further, in Embodiment 2, an irradiation position stage 9c, which is a unit to parallel-shift the emitting end 3h, is disposed inside the probe 101.

Furthermore, in Embodiment 2, the camera 8a is disposed at a position which allows imaging a region in the scanning range of the scanning stage 9a.

In Embodiment 2, in a case where the irradiation position of the light becomes close to the high absorption region during scanning, the irradiation position stage 9c is driven and the position of the emitting end 3h is temporarily shifted (shifted relative to the acoustic wave probe 1), so as to avoid the high absorption region.

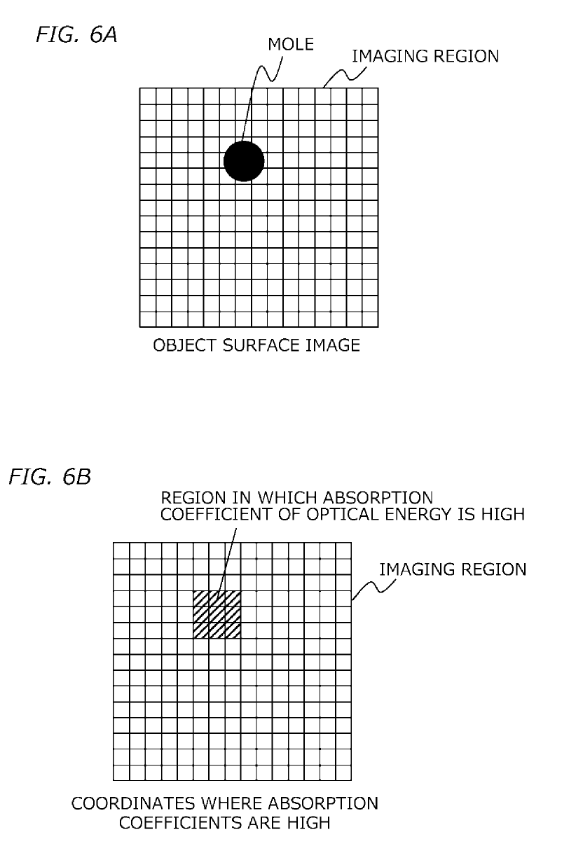

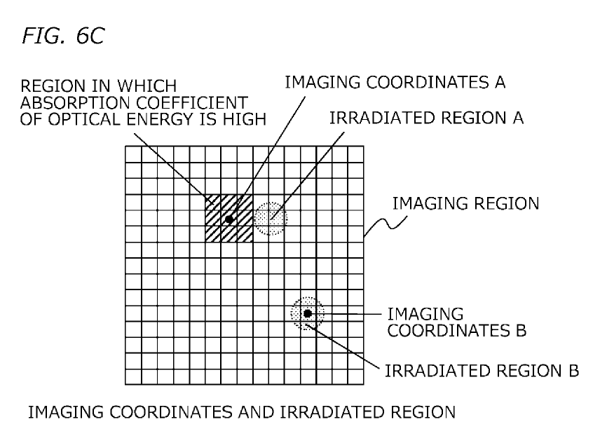

Details of the control performed by the control unit 6 according to Embodiment 2 will be described with reference to FIG. 6A to FIG. 6C.

FIG. 6A to FIG. 6C illustrate imaging regions where the grid indicates the imaging coordinates. If there is a mole on the surface within the imaging region, an image illustrated in FIG. 6A is captured by the camera 8a.

In the case of this example, the control unit 6 records that the high absorption region exists in the region hatched (“a first region”) in FIG. 6B.

Here a case where the probe 101 is moved by the scanning stage 9a to perform measurement in the imaging coordinates indicated in FIG. 6C will be described. It is assumed that, in the first region including imaging coordinates A, the intensity of the irradiated light to the high absorption region exceeds a predetermined value. In this case, the control unit 6 controls the position of the irradiation position stage 9c to temporarily parallel-shift the irradiated region, so that the intensity of the light irradiated to the high absorption region does not exceed a predetermined value. In the case of this example, the light is irradiated to the region indicated as the irradiated region A (“a second region”).

In the case of performing measurement in the imaging coordinates B, on the other hand, the control unit 6 does not shift the irradiated region. As a result, the light is irradiated to the irradiated region B.

According to Embodiment 2, the irradiation position of the light is shifted in a single direction, therefore effects similar to Embodiment 1 can be implemented without changing the intensity distribution of the irradiated light. Thereby the later mentioned correction of the light quantity distribution can be performed accurately. Further, compared with Embodiment 1, the light can be irradiated more closely to the high absorption region, hence the irradiated light can reach more strongly to a region deeper than the high absorption region. In other words, information on the deep region of the object can be acquired more accurately.

### Embodiment 3

In Embodiments 1 and 2, the irradiation position of the light is adjusted by moving the irradiation position stage 9b or 9c. In Embodiment 3, on the other hand, the irradiation position of the light is adjusted by shielding part of the irradiated light.

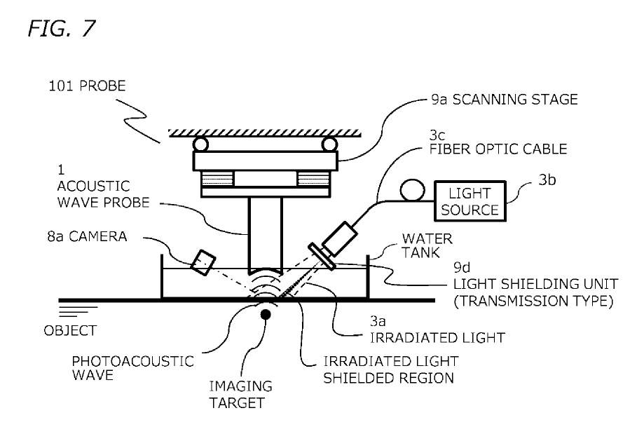

FIG. 7 is a block diagram depicting a photoacoustic microscope according to Embodiment 3.

The photoacoustic microscope according to Embodiment 3 is the same as the photoacoustic microscope according to Embodiment 2, except that the irradiation position stage 9c is omitted and a light shielding unit 9d is added. The light shielding unit 9d is a unit that shields at least a part of the irradiated light 3a irradiated from the emitting end (light shielding member).

In Embodiment 3, in a case where the irradiation position of the light is not close to the high absorption region during scanning, the irradiated light 3a is irradiated to a position facing the acoustic wave probe 1. At this time, the irradiation range of the irradiated light is approximately the same as the reception range of the acoustic wave probe 1. When the irradiation position of the light is close to the high absorption region, on the other hand, the light shielding unit 9d is driven to shield a part of the irradiated light, so that the intensity of the light irradiated to the high absorption region does not exceed a predetermined value.

For the light shielding unit 9d, a transmission type liquid crystal device, for example, may be used. In this case, a part of the irradiated light can be shielded by turning the pixels ON that correspond to the region to be shielded.

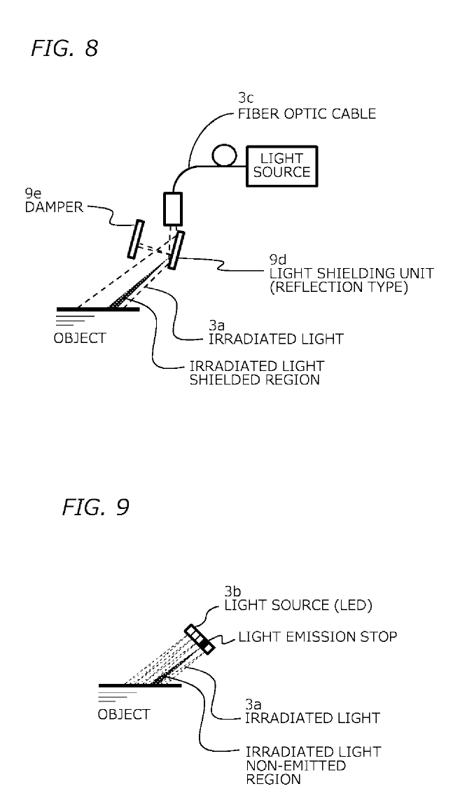

The light shielding unit 9d may be a different device. For example, as illustrated in FIG. 8, a reflection type device, such as a digital mirror array, may be used. In this case, the pixels that correspond to the region to be shielded are driven, whereby the light that enters these pixels can be reflected to a damper 9e.

Not only shielding the irradiated light but also a light emitting element array may be used for the light source, so that emission of desired elements can be stopped. For example, as illustrated in FIG. 9, light emitting array elements, such as an LED, may be used for the light source 3b. In this case, the control unit 6 may change the light emitting state of the target elements (e.g. stopping light emission, decreasing light quantity).

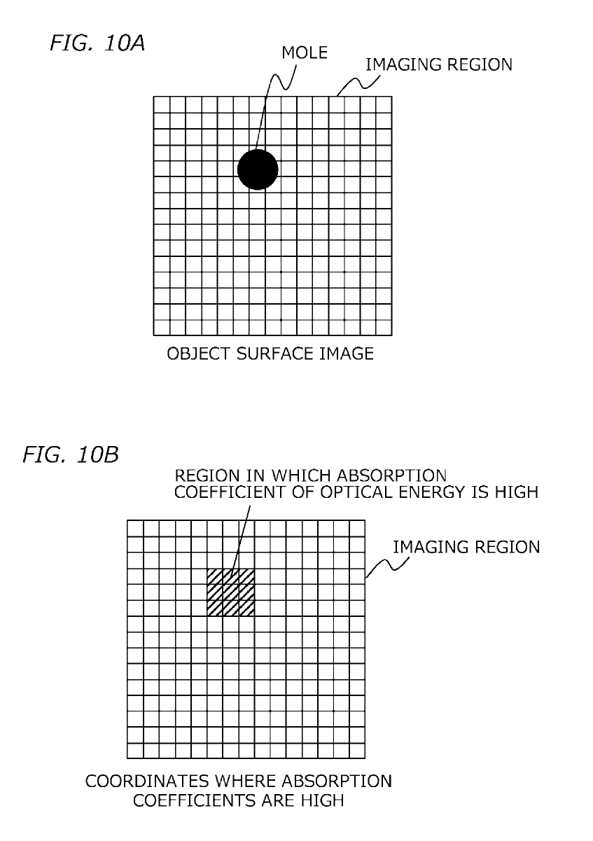

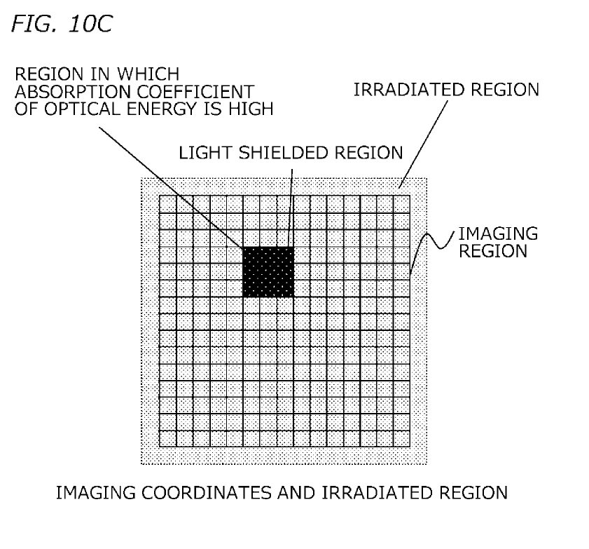

Details of the control performed by the control unit 6 according to Embodiment 3 will be described with reference to FIG. 10A to FIG. 10C.

FIG. 10A to FIG. 10C illustrate the imaging regions, where the grid indicates the imaging coordinates. If there is a mole on the surface within the imaging region, an image illustrated in FIG. 10A is captured by the camera 8a.

In the case of this example, the control unit 6 records that the high absorption region exists in the region hatched in FIG. 10B.

Here a case of imaging the imaging region illustrated in FIG. 10C will be described. In this case, the control unit 6 controls and shields light in the region indicated by black, so that the intensity of light irradiated to the high absorption region does not exceed a predetermined value. In the case of this example, light is irradiated to the irradiated region excluding the black region. In this state, the acoustic wave probe 1 performs imaging while scanning the imaging region.

According to Embodiment 3, irradiation of the light to the high absorption region can be avoided without changing the shape of the irradiated light or without temporarily shifting the irradiation position. In other words, the irradiation position can be adjusted by a simpler control than Embodiments 1 and 2.

### Embodiment 4

In Embodiments 1 to 3, the high absorption region is extracted based on the image acquired by imaging the object surface. In Embodiment 4, on the other hand, the high absorption region is extracted using a photoacoustic image acquired in advance.

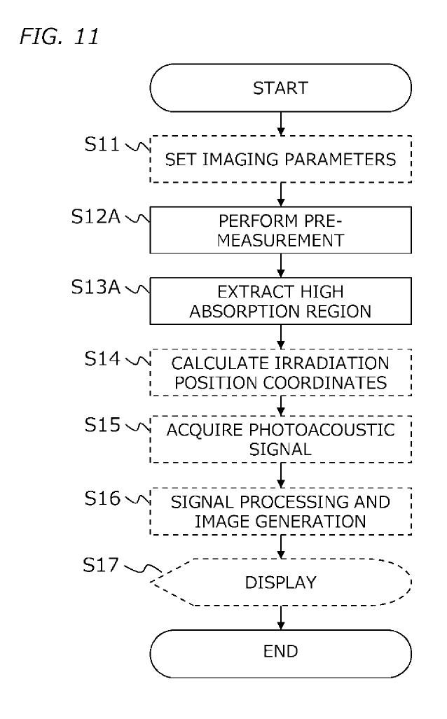

The configuration of a photoacoustic microscope according to Embodiment 4 is the same as Embodiments 1 to 3, except that the object observing unit 8 (camera 8a) is not essential. A processing flow performed by the photoacoustic microscope according to Embodiment 4 will be described next with reference to FIG. 11.

According to Embodiment 4, in step S12A, light is irradiated to the object, and the photoacoustic signal is acquired. In this step, the control unit 6 acquires the photoacoustic signal by the same method as step S15 using the imaging parameters acquired in step S11. The position of the high absorption region is unknown, hence in step S12A, the light is irradiated to a predetermined position, including the object. Then the signal is processed by the same method as step S16 in order to generate the image.

In the following description, the processing performed in step S12A is called a “pre-measurement”.

The resolution of the image generated in the pre-measurement may be lower than that of the main measurement. Further, the range to be image may be limited to a shallow portion of the object. Thereby the time required for the pre-measurement can be decreased.

In step S13A, the high absorption region is extracted based on the photoacoustic image acquired in the pre-measurement. When the photoacoustic measurement is performed without considering the high absorption region, a strong acoustic wave is generated from a region where a mole or a nevus exists, therefore the brightness of this region becomes high in the acquired photoacoustic image. In other words, the high absorption region can be extracted by binarizing the photoacoustic image acquired in the pre-measurement, using a predetermined threshold.

The subsequent steps are the same as the steps described with reference to FIG. 4, hence description thereof is omitted.

According to Embodiment 4, the high absorption region can be extracted without using a camera for imaging the object surface. Further, even a portion that is difficult to recognize in the image of the object surface, such as a thick subcutaneous blood vessel, can be extracted as the high absorption region. According to Embodiment 4, these portions which are not really necessary to evaluate the pathological state can be efficiently removed.

### Embodiment 5

In Embodiments 1 to 3, the high absorption region is extracted based on the image acquired by imaging the object surface in advance. In Embodiment 5, on the other hand, the irradiation position is controlled in real-time while observing the object surface during measurement, without extracting the high absorption region in advance.

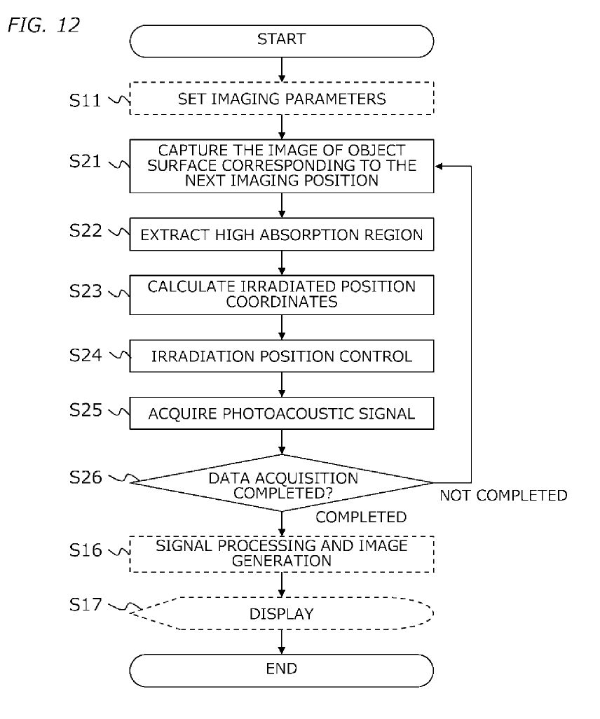

The processing flow performed by the photoacoustic microscope according to Embodiment 5 will be described with reference to FIG. 12.

According to Embodiment 5, in a case where imaging of an object is performed for a plurality of times while shifting the imaging position, an image of the object surface is acquired using the camera 8a during an interval of the imaging (step S21). In this step, the irradiated light 3a is stopped, and the scanning stage 9a is moved to the next imaging position, and imaging is then performed by the camera 8a. Here the position to which the scanning stage 9c is moved is not limited to “the next imaging position”. For example, the scanning stage 9c may be moved to a second or later imaging position after next imaging.

In steps S22 and S23, the high absorption region is extracted based on the image acquired in step S21, and the coordinates of the irradiation position are calculated and recorded. The coordinates recorded here are coordinates where the light is irradiated during the next imaging.

Then in step S24, the irradiation position in the next imaging is controlled in accordance with the coordinates recorded in step S23.

Then in step S25, light is irradiated to the object, and the photoacoustic signal is acquired.

Then in step S26, it is determined whether or not the acquisition of the data completed (that is, whether or not imaging completed for all imaging positions), and if not completed, processing advances to step S21. If the acquisition of the data is completed, processing advances to step S16.

As described above, according to Embodiment 5, the high absorption region is extracted in real-time while measurement is performed. Thereby the time required for the entire imaging of the photoacoustic image can be decreased.

### Embodiment 6

In Embodiments 1 to 5, the irradiation position of the light to the object is changed based on the extracted high absorption region. However if the irradiation position is changed, the light quantity distribution inside the object changes, which may result in a drop in the accuracy of the object information.

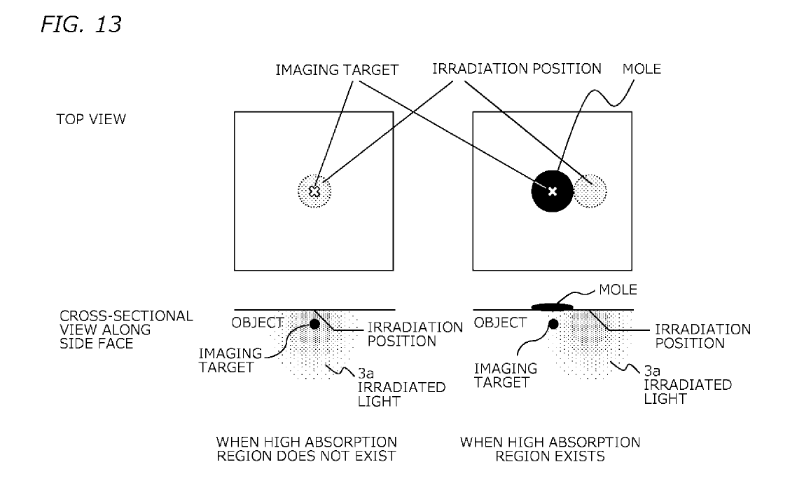

FIG. 13 indicates top views and cross-sectional views along the side face of the object. The white X symbol indicates the position of the imaging target existing in the object.

If a high absorption region does not exist on the surface of the object, the irradiation position is determined so as to include the imaging target, as illustrated on the left side in FIG. 13. If a high absorption region exists on the surface of the object, on the other hand, the irradiation position is determined so as to avoid this region, as illustrated on the right side in FIG. 13 (this is the case of Embodiment 2).

The density of the dots in the cross-sectional views indicates the intensity of the irradiated light which was diffused inside the object. As illustrated here, if the irradiation position changes, the light quantity changes inside the object also changes, therefore the intensity of the photoacoustic wave emitted from the same light absorber also changes. In other words, the quantitativeness of the acquired photoacoustic image may be diminished.

Therefore in Embodiment 6, the light quantity distribution inside the object is corrected, and the quantitativeness of the photoacoustic image is improved.

In concrete terms, the control unit 6 transfers both the imaging position coordinates of the object and the irradiation position coordinates to the processing unit 2. Then the processing unit 2 calculates the light quantity distribution of the light irradiated to the object surface for each irradiation position coordinate, and uses this result to generate the photoacoustic image.

The light quantity distribution inside the object can be acquired by a transport diffusion equation or the Monte Carlo method based on the average absorption coefficient or scattering coefficient inside the object and the light quantity distribution of the light irradiated to the object surface. Therefore the light quantity distribution inside the object can be accurately determined, whether the imaging position and the irradiation position are the same or not.

The sound pressure of the photoacoustic wave is a product of a Griineisen coefficient, the absorption coefficient of the imaging target, and the light quantity. This means that the absorption coefficient can be determined at a higher precision by accurately determining the light quantity inside the object. Further, the precision of determining the oxygen saturation, the plaque inside a blood vessel and the like can be improved since these values can be calculated based on the absorption coefficient acquired for each wavelength.

In this way, according to Embodiment 6, the quantitativeness of the acquired photoacoustic image can be improved by correcting (regenerating) the light quantity distribution.

Here the method of determining the light distribution intensity using a transport diffusion equation or the Monte Carlo method based on the average absorption coefficient or scattering coefficient inside the object and the light distribution intensity of the light irradiated to the object surface was described. But the method of determining the light distribution intensity is not limited to this, and it is also effective if the processing unit 2 uses a simple equation or a table, so that the light distribution intensity is determined referring to either the equation or the table, and the light quantity is corrected. Thereby calculation for the light quantity correction can be performed for a short time.

### Embodiment 7

In Embodiments 1 to 6, the photoacoustic microscope was described as an example, but the present invention may be applied to photoacoustic mammography and the like. Particularly in the case where the object is a breast, not only a mole but also an area around the nipple becomes a region having a high absorption coefficient, hence the present invention can be suitably applied.

In Embodiment 7, a probe in which acoustic elements are arrayed in a 1D array, 2D array, hemispherical array or the like is used for the acoustic wave probe 1. Embodiment 7 can be applied in particularly to CT, such as photoacoustic mammography for breasts.

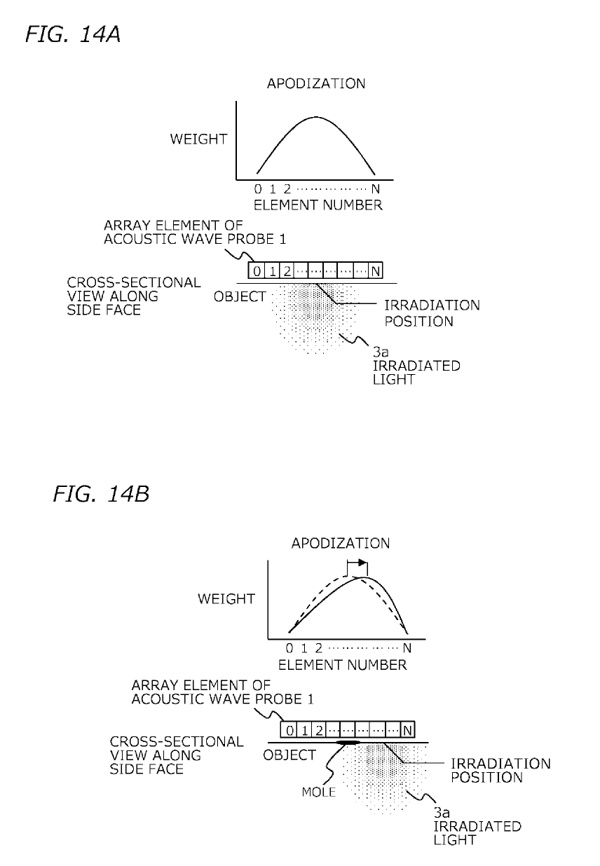

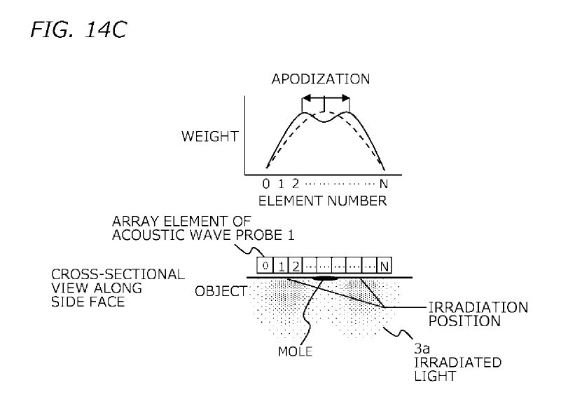

In the case of using an acoustic wave probe in which acoustic elements are arrayed, the acoustic elements are normally weighted (apodized) in accordance with the irradiation position of the light. For example, the arrayed acoustic elements are apodized in accordance with a window function, such as the Hanning function, as illustrated in FIG. 14A. In a normally performed apodization, a larger weight is assigned as the element is disposed closer to the center among the arrayed elements.

In the case where there is a high absorption region on the object surface, on the other hand, light is irradiated so as to exclude this region, as illustrated in FIG. 14B. In this case, a large weight is assigned to the acoustic elements that are closer to the irradiation position than in a normal case. In the case where the light is irradiated to a ring-shaped region, as illustrated in FIG. 14C, as well, a large weight is assigned to the acoustic elements that are closer to the irradiation position. In this way, apodization may be expressed by a high order function.

In this way, by performing apodization in accordance with the irradiation position, a larger weight can be assigned as the element is closer to the generation source of the acoustic wave. In other words, contrast can be improved in a case where the photoacoustic signal is imaged.

In a case where apodization is performed, the weights to elements that are distant from the irradiation position on the object surface for at least a predetermined value may be set to zero or to a very low value. In other words, by eliminating the influence of the receive signals from the elements that are distant from the generation source of the photoacoustic wave emitted from a region where irradiated light is strong, contrast can be further improved.

Modifications

The above description on each embodiment is an example to describe the present invention, and the present invention can be carried out by appropriately changing or combining the above embodiments within a scope that does not depart from the essence of the invention.

For example, the present invention may be carried out as a photoacoustic apparatus that includes at least a part of the above mentioned units. The present invention may also be carried out as an object information acquiring method that includes at least a part of the above mentioned processing. The above processing and units may be freely combined within the scope of not generating technical inconsistencies.

In the description of the embodiments, an apparatus that performs only photoacoustic measurement was described as an example, but a function to perform ultrasonic (ultrasonic echo) measurement may be added to the photoacoustic apparatus 110. For example, the acoustic wave probe 1 may transmit the ultrasonic wave to an object and receive the reflected wave thereof, and the processing unit 2 may generate an ultrasonic image based on the received reflected wave. Further, if the acoustic wave probe 1 has a plurality of transducers, the processing unit 2 may perform beam forming processing.

In the case of using both the photoacoustic measurement and the ultrasonic measurement, the timing of emitting the light from the light source 3b and the timing of transmitting the ultrasonic wave from the acoustic wave probe 1 must be separated, so that the photoacoustic wave and the ultrasonic echo do not interfere with each other inside the living body.

Therefore, for example, images are normally generated by transmitting and receiving the ultrasonic wave in real-time, and in a case where the user performs an operation, the transmission/reception of the ultrasonic waves is stopped, and the mode is shifted to photoacoustic mode.

When the ultrasonic image is acquired, any imaging mode may be selected from: B-mode tomography, color doppler, power doppler and the like. Further, the focus setting inside the object and other information may be acquired from an outside source, and the processing unit 2 may perform beam forming and generate an image in accordance with the setting content.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-217509, filed on Nov. 10, 2017, which is hereby incorporated by reference herein in its entirety.

What is claimed is:

1. A photoacoustic apparatus configured to receive an acoustic wave generated from an object to which light is irradiated, and to generate object information which is information on the object, the apparatus comprising:
an irradiation control unit configured to control an irradiated region of the light on the object;
an acoustic wave receiving unit configured to receive the acoustic wave generated from the object, and to convert the acoustic wave into a received signal;
a generating unit configured to generate the object information based on the received signal; and
a setting unit configured to set, for the object, a high absorption region in which a value related to absorption of an optical energy is at least a predetermined value, based on the received signal,
wherein the irradiation control unit controls the irradiated region of the light on the object based on a position of the high absorption region.

2. The photoacoustic apparatus according to claim 1, wherein the irradiation control unit controls the irradiated region of the light on the object so that the high absorption region is not irradiated with light having intensity of at least a predetermined value.

3. The photoacoustic apparatus according to claim 1, wherein in a case where intensity of light irradiated to the high absorption region in a first region exceeds a predetermined value, and intensity of light irradiated to the high absorption region in a second region becomes less than the predetermined value, the irradiation control unit shifts the irradiated region of the light to the second region.

4. The photoacoustic apparatus according to claim 3, wherein the generating unit generates a light quantity distribution, inside the object, of the light irradiated to the irradiated region of the light and generates the object information based on the light quantity distribution.

5. The photoacoustic apparatus according to claim 3, wherein the acoustic wave receiving unit includes a plurality of acoustic elements which are disposed in an array, and weights each of signals output from the plurality of acoustic elements, based on the irradiated region of the light.

6. The photoacoustic apparatus according to claim 1, wherein the setting unit sets the high absorption region based on an image capturing a surface of the object.

7. The photoacoustic apparatus according to claim 6, wherein the setting unit extracts, from the image, a region having a density exceeding a predetermined threshold, and sets the region as the high absorption region.

8. The photoacoustic apparatus according to claim 1,
wherein the light is irradiated to the object via an optical system which can be moved by a scanning unit, and
the irradiation control unit controls the irradiated region of the light on the object by moving the optical system using the scanning unit.

9. The photoacoustic apparatus according to claim 1,
wherein the light is irradiated to the object via an optical system which irradiates the light to a ring-shaped region, and
the irradiation control unit controls the irradiated region of the light on the object by changing an inner diameter and an outer diameter of the ring.

10. The photoacoustic apparatus according to claim 1, wherein the irradiation control unit controls the irradiated region of the light on the object using a light shielding member.

11. The photoacoustic apparatus according to claim 1,
wherein the light is generated by a light emitting element array, and
the irradiation control unit controls the irradiated region of the light on the object by changing light emitting states of a plurality of elements included in the light emitting element array.

12. An object information acquiring method performed by a photoacoustic apparatus configured to receive an acoustic wave generated from an object to which light is irradiated, and to generate object information which is information on the object, the method comprising:
an irradiation control step of controlling an irradiated region of the light on the object;
an acoustic wave receiving step of receiving the acoustic wave generated from the object, and converting the acoustic wave into a received signal;
a generating step of generating the object information based on the received signal; and
a setting step of setting, for the object, a high absorption region in which a value related to absorption of an optical energy is at least a predetermined value, based on the received signal,
wherein, in the irradiation control step, the irradiated region of the light on the object is controlled based on a position of the high absorption region.
